(19) United States
(12) Patent Application Publication
(10) Pub. No.: US 2025/0153164 A1
(43) Pub. Date: May 15, 2025

(54) HYDROGEL CHANNEL DEVICE WITH SENSOR

(71) Applicant: NIPPON TELEGRAPH AND TELEPHONE CORPORATION, Tokyo (JP)

(72) Inventors: Aya TANAKA, Musashino-shi, Tokyo (JP); Riku TAKAHASHI, Musashino-shi, Tokyo (JP); Suzuyo INOUE, Musashino-shi, Tokyo (JP); Tomomi MURAI, Musashino-shi, Tokyo (JP); Masumi YAMAGUCHI, Musashino-shi, Tokyo (JP)

(73) Assignee: NIPPON TELEGRAPH AND TELEPHONE CORPORATION, Tokyo (JP)

(21) Appl. No.: 18/838,182

(22) PCT Filed: Feb. 22, 2022
(86) PCT No.: PCT/JP2022/007408

§ 371 Date: Aug. 13, 2024

(51) Int. Cl. B01L 3/00; G01N 33/543

(52) U.S. Cl. CPC B01L 3/5027; G01N 33/543

(57) ABSTRACT

A hydrogel channel type device (1) with a sensor according to the present invention includes: a solid substrate (10) and a hydrogel laminate (20) located on the solid substrate (10), in which the solid substrate (10) has an adhered region that is adhered to the hydrogel laminate (20) and a non-adhered region that is not adhered to the hydrogel laminate (20) at an interface with the hydrogel laminate (20), the hydrogel laminate (20) includes a swellable gel thin film layer (21) on the solid substrate (10) and a non-swellable gel layer (22) laminated on the swellable gel thin film layer (21), a hydrogel channel (30) in which the swellable gel thin film layer (21) is separated in the non-adhered region is provided between the solid substrate (10) and the hydrogel laminate (20), and a sensor section is provided at the interface between the solid substrate (10) and the hydrogel laminate (20).

### TECHNICAL FIELD

The present invention relates to a hydrogel channel type device with a sensor.

### BACKGROUND ART

In recent years, attempts have been made to simulate biological functions and structures in vitro using a microfluidic chip in drug discovery research for the purpose of disease treatment and research to elucidate the mechanism of disease onset. As an attempt, for example, it is known to construct a model system called a biomimetic system (hereinafter abbreviated as an “MPS”). Microfluidic chips are widely used in an MPS. As a base material of an MPS, a base material made of a material capable of culturing cells and having easy moldability is used. Examples of such a material include glass, polymethyl methacrylate (PMMA), and polydimethylsiloxane (PDMS).

On the other hand, in an MPS, it is necessary to understand the evaluation of actions of oral drugs and the dynamics when causative substances of infectious diseases and diseases act on the disease site. To this end, it is necessary to evaluate the diffusion and actions of drugs and causative substances reaching the site of action of living tissues from circulatory systems including blood vessels.

Materials used in conventional microfluidic devices have low substance permeability and are not suitable for the above purposes. Therefore, a material suitable for the above purpose is required.

In order to achieve the above purpose, it is known that hydrogels having high substance permeability and biocompatibility are effective. As a microfluidic device using a hydrogel, for example, a microfluidic device in which adhered/non-adhered regions between a solid substrate and a swellable gel are disposed in a pattern and which has a three-dimensional structure due to free swelling of the hydrogel in the non-adhered region is known (see, for example, Patent Literature 1). In addition, a hydrogel channel type device using a three-dimensional structure as a channel is known (see, for example, Patent Literature 2). Furthermore, it is known that the hydrogel channel type device can be used as a substance permeation type microfluidic device, such as for diffusion of a dye from a channel, cell culture on the hydrogel channel type device, and drug stimulation of cells (see, for example, Non Patent Literature 1).

### CITATION LIST

### Patent Literature

Patent Literature 1: JP 2020-62843 A
Patent Literature 2: WO 2021/079399 A

### Non Patent Literature

Non Patent Literature 1: “Tough, permeable and biocompatible microfluidic devices formed through the buckling delamination of soft hydrogel films”, Riku Takahashi, et al., The Royal Society of Chemistry 2021, Lab Chip, 2021, 21, 1307-1317

### SUMMARY OF INVENTION

### Technical Problem

In the substance permeation by the hydrogel channel type device, it is essential to use a dye in order to visualize the substance diffusion into the device over time. On the other hand, when pharmacodynamic analysis by an MPS or the dynamics of a disease-causing substance is measured, it is often impossible to visualize a target drug or causative substance (hereinafter referred to as an “analyte”). Examples of a method for visualizing an analyte include a method in which the analyte is modified with a labeling agent (fluorescent dyes, nanoparticles, etc.). However, this method has problems in that it is complicated and that modification with a labeling agent is likely to change the permeability and diffusion rate of the target substance.

The present invention has been made in view of the above circumstances, and an object of the present invention is to provide a hydrogel channel type device with a sensor capable of producing a tissue-like structure simulating the inside of a biological environment on a chip outside a living body and measuring molecular diffusion outside the structure through a blood-vessel-like tubular structure.

### Solution to Problem

One aspect of the present invention is a hydrogel channel type device with a sensor, the device including: a solid substrate and a hydrogel laminate located on the solid substrate, in which the solid substrate has an adhered region that is adhered to the hydrogel laminate and a non-adhered region that is not adhered to the hydrogel laminate at an interface with the hydrogel laminate, the hydrogel laminate includes a swellable gel thin film layer on the solid substrate and a non-swellable gel layer laminated on the swellable gel thin film layer, a hydrogel channel in which the swellable gel thin film layer is separated in the non-adhered region is provided between the solid substrate and the hydrogel laminate, and a sensor section is provided at the interface between the solid substrate and the hydrogel laminate.

### Advantageous Effects of Invention

According to the present invention, it is possible to provide a hydrogel channel type device with a sensor capable of producing a tissue-like structure simulating the inside of a biological environment on a chip outside a living body and measuring molecular diffusion outside the structure through a blood-vessel-like tubular structure.

### BRIEF DESCRIPTION OF DRAWINGS

FIG. 1 is a perspective view illustrating a schematic configuration of a hydrogel channel type device with a sensor according to an embodiment of the present invention.

FIG. 2 is a perspective view illustrating a schematic configuration of a solid substrate and a hydrogel laminate constituting the hydrogel channel type device with a sensor according to the embodiment of the present invention.

FIG. 3 is a plan view illustrating a schematic configuration of the solid substrate constituting the hydrogel channel type device with a sensor according to the embodiment of the present invention.

FIG. 4 is a side view illustrating a schematic configuration of the solid substrate constituting the hydrogel channel type device with a sensor according to the embodiment of the present invention.

FIG. 5 is an enlarged view of a region x illustrated in FIG. 4, illustrating a schematic configuration of the solid substrate constituting the hydrogel channel type device with a sensor according to the embodiment of the present invention.

FIG. 6 is an enlarged view of a region x illustrated in FIG. 4, illustrating a schematic configuration of the solid substrate constituting the hydrogel channel type device with a sensor according to the embodiment of the present invention.

FIG. 7 is a side view illustrating the concept of diffusion of an analyte solution by a hydrogel channel type device with an SPR sensor and detection by the SPR sensor.

FIG. 8 is a side view illustrating the concept of diffusion of an analyte solution by a hydrogel channel type device with an SPR sensor and detection by the SPR sensor.

FIG. 9 is a side view illustrating the concept of diffusion of an analyte solution by a hydrogel channel type device with an SPR sensor and detection by the SPR sensor.

FIG. 10 is a side view illustrating the concept of diffusion of an analyte solution by a hydrogel channel type device with an SPR sensor and detection by the SPR sensor.

FIG. 11 is a view illustrating the relationship between a diffusion position of an analyte solution with respect to a hydrogel laminate and a diffusion time of the analyte solution with respect to the hydrogel laminate.

FIG. 12 is a view illustrating the relationship between a diffusion time of an analyte solution with respect to a hydrogel laminate and an intensity of an absorption spectrum of light on a detection surface of a sensor section.

FIG. 13 is a side view illustrating the concept of diffusion of an analyte solution by a hydrogel channel type device with a graphene/DNA aptamer sensor and detection by the graphene/DNA aptamer sensor.

FIG. 14 is a side view illustrating the concept of diffusion of an analyte solution by a hydrogel channel type device with a graphene/DNA aptamer sensor and detection by the graphene/DNA aptamer sensor.

FIG. 15 is a side view illustrating the concept of diffusion of an analyte solution by a hydrogel channel type device with a graphene/DNA aptamer sensor and detection by the graphene/DNA aptamer sensor.

FIG. 16 is a side view illustrating the concept of diffusion of an analyte solution by a hydrogel channel type device with a graphene/DNA aptamer sensor and detection by the graphene/DNA aptamer sensor.

FIG. 17 is a view illustrating the relationship between a diffusion position of an analyte solution with respect to a hydrogel laminate and a fluorescence intensity of a fluorescent dye at the end of a DNA aptamer.

FIG. 18 is a plan view illustrating a schematic configuration of a modification example of the solid substrate constituting the hydrogel channel type device with a sensor according to the embodiment of the present invention.

FIG. 19 is a side view illustrating a schematic configuration of the modification example of the solid substrate constituting the hydrogel channel type device with a sensor according to the embodiment of the present invention.

FIG. 20 is a view illustrating the results of measuring the diffusion of a rhodamine solution in a hydrogel channel type device with an SPR sensor using the SPR sensor in Examples.

### DESCRIPTION OF EMBODIMENTS

Hereinafter, a hydrogel channel type device with a sensor according to an embodiment to which the present invention is applied will be described in detail with reference to the drawings. Note that, in the drawings used in the following description, in order to facilitate understanding of characteristics, a characteristic portion may be illustrated in an enlarged manner for convenience, and dimensional ratios and the like of components are not necessarily the same as actual ones. In addition, materials, dimensions, and the like exemplified in the following description are merely examples, and the present invention is not limited thereto, and can be appropriately changed and implemented without changing the gist thereof.

[Hydrogel Channel Type Device with Sensor]

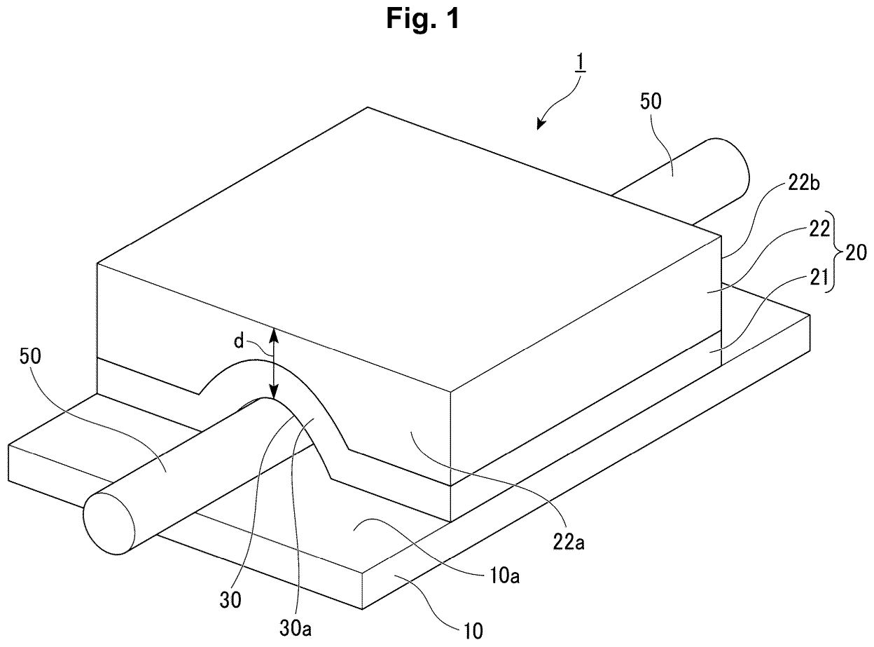

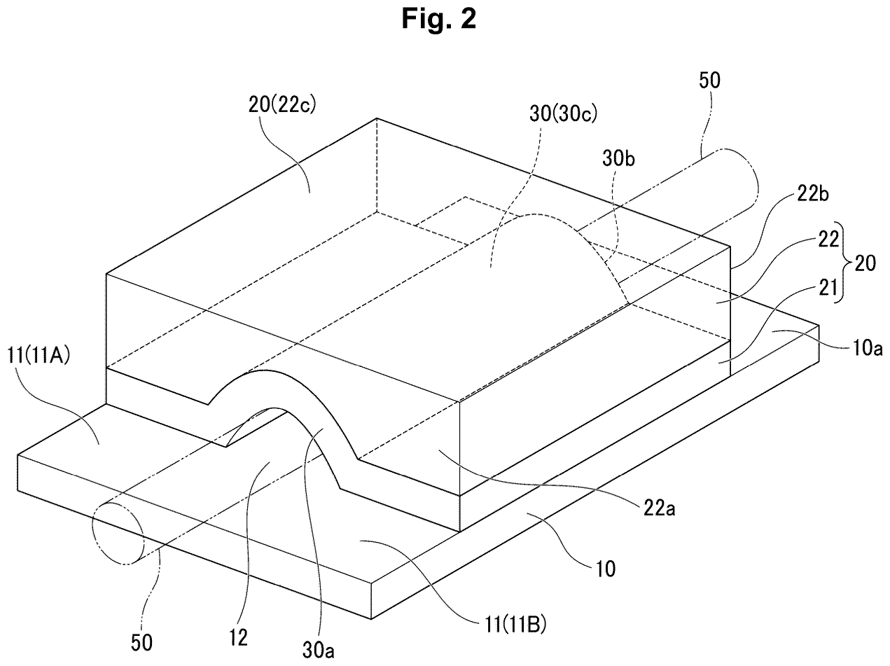

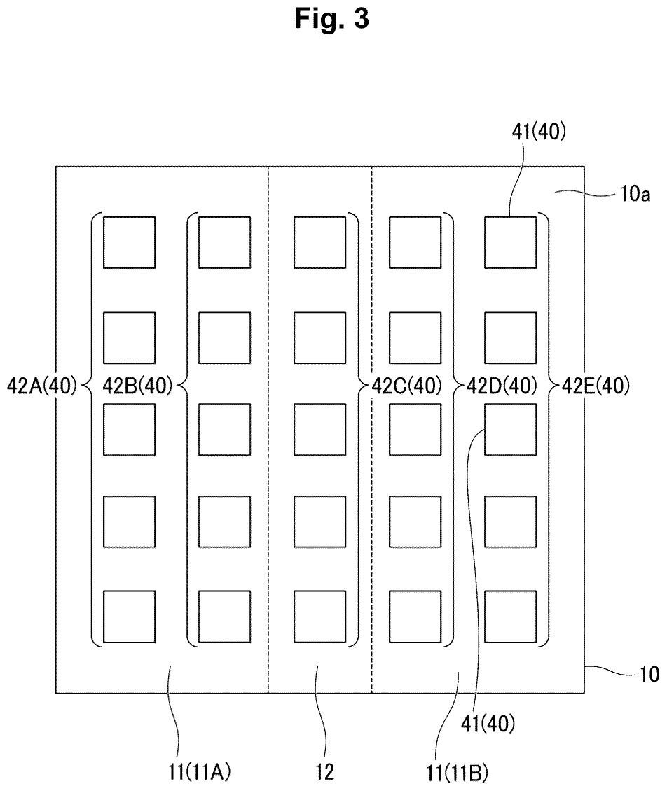

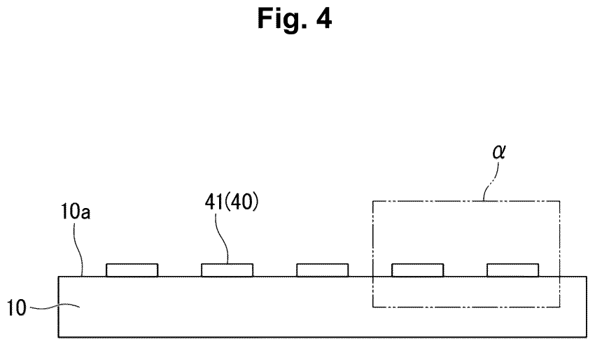

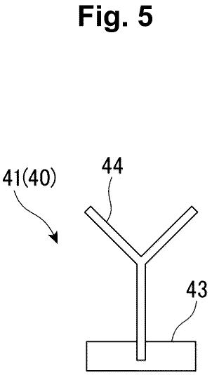

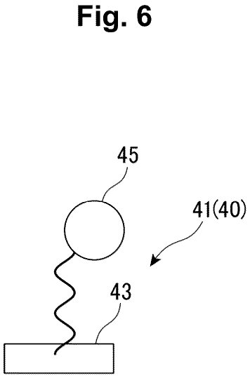

FIG. 1 is a perspective view illustrating a schematic configuration of a hydrogel channel type device with a sensor according to an embodiment of the present invention. FIG. 2 is a perspective view illustrating a schematic configuration of a solid substrate and a hydrogel laminate constituting the hydrogel channel type device with a sensor according to the embodiment of the present invention. FIG. 3 is a plan view illustrating a schematic configuration of the solid substrate constituting the hydrogel channel type device with a sensor according to the embodiment of the present invention. FIG. 4 is a side view illustrating a schematic configuration of the solid substrate constituting the hydrogel channel type device with a sensor according to the embodiment of the present invention. FIGS. 5 and 6 are enlarged views of a region a illustrated in FIG. 4, illustrating a schematic configuration of the solid substrate constituting the hydrogel channel type device with a sensor according to the embodiment of the present invention.

As illustrated in FIGS. 1 to 4, a hydrogel channel type device 1 with a sensor of the present embodiment includes a solid substrate 10 and a hydrogel laminate 20 located on the solid substrate 10. As illustrated in FIG. 2, the solid substrate 10 has adhered regions 11 that are adhered to the hydrogel laminate 20 and a non-adhered region 12 that is not adhered to the hydrogel laminate 20 at an interface with the hydrogel laminate 20. Here, the interface between the solid substrate 10 and the hydrogel laminate 20 is one surface (upper surface illustrated in FIGS. 1 to 4) 10a of the solid substrate 10. As illustrated in FIGS. 1 and 2, the hydrogel laminate 20 includes a swellable gel thin film layer 21 on one surface 10a of the solid substrate 10 and a non-swellable gel layer 22 laminated on the swellable gel thin film layer 21. The swellable gel thin film layer 21 adheres to the adhered region 11 and does not adhere to the non-adhered region 12. The hydrogel channel type device 1 with a sensor includes a hydrogel channel 30 in which the swellable gel thin film layer 21 is separated in the non-adhered region 12 between the solid substrate 10 and the hydrogel laminate 20. As illustrated in FIGS. 3 and 4, the hydrogel channel type device 1 with a sensor includes a sensor section 40 at the interface between the solid substrate 10 and the hydrogel laminate 20. As illustrated in FIG. 1, the hydrogel channel type device 1 with a sensor preferably includes a liquid feeding tube 50 connected to the hydrogel channel 30.

### “Solid Substrate”

The solid substrate 10 supports the hydrogel laminate 20.

The solid substrate 10 is not particularly limited as long as the effect of the present invention is not impaired, and examples thereof include a substrate made of an inorganic material such as glass or silicon, and a plastic film made of an organic material such as polysilicone or polyurethane. As the solid substrate 10, one whose one surface 10a is covered with a thin film made of a metal or an inorganic oxide having an arbitrary function or a thin film of an arbitrary shape made of an organic material having an arbitrary function can be used.

The adhered region 11 has a first adhered region 11A and a second adhered region 11B with the non-adhered region 12 in between.

The adhered region 11 and the non-adhered region 12 have a band shape with one side as a long side in a plan view. In FIG. 1, an X direction is defined as a long side (length) of the adhered region 11 and the non-adhered region 12, and a Y direction is defined as a short side (width) of the adhered region 11 and the non-adhered region 12. The long side of the solid substrate 10 and the hydrogel laminate 20 is in the same direction as the long side of the adhered region 11 and the non-adhered region 12. In addition, the short side of the solid substrate 10 and the hydrogel laminate 20 is in the same direction as the short side of the adhered region 11 and the non-adhered region 12. The non-adhered region 12 is disposed in a band shape inside the hydrogel channel 30. The adhered regions 11 are disposed on both sides of the non-adhered region 12 in the extending direction.

### (Sensor Section)

The sensor section 40 is provided on the adhered region 11 and the non-adhered region 12. The sensor section 40 includes a plurality of unit sensor section rows 42 each including two or more unit sensor sections 41 arranged in the length direction of the adhered region 11 and the non-adhered region 12. Here, a case where the sensor section 40 includes a first unit sensor section row 42A, a second unit sensor section row 42B, a third unit sensor section row 42C, a fourth unit sensor section row 42D, and a fifth unit sensor section row 42E each including the unit sensor sections 41 will be exemplified. In addition, the first unit sensor section row 42A, the second unit sensor section row 42B, the third unit sensor section row 42C, the fourth unit sensor section row 42D, and the fifth unit sensor section row 42E are arranged apart from each other in the width direction of the adhered region 11 and the non-adhered region 12. The sensor section 40 includes a plurality of unit sensor sections 41 provided on one surface 10a of the solid substrate 10 to be separated from each other. In other words, the sensor section 40 is a set of a plurality of unit sensor sections 41 provided on one surface 10a of the solid substrate 10. The unit sensor sections 41 constitute the first unit sensor section row 42A, the second unit sensor section row 42B, the third unit sensor section row 42C, the fourth unit sensor section row 42D, and the fifth unit sensor section row 42E. In addition, the unit sensor sections 41 are discontinuously present in the length direction of one surface 10a of the solid substrate 10 in a plan view. In the present embodiment, “discontinuous” means that the unit sensor section 41 has an island-like structure, and there is a portion where the solid substrate 10 is exposed on one surface 10a of the solid substrate 10.

In the adhered region 11, the hydrogel laminate 20 (swellable gel thin film layer 21) is adhered to one surface 10a of the solid substrate 10. Therefore, the sensor section 40 is covered with the hydrogel laminate 20 (swellable gel thin film layer 21).

On the other hand, in the non-adhered region 12, the hydrogel laminate 20 (swellable gel thin film layer 21) is not adhered to one surface 10a of the solid substrate 10. Therefore, the sensor section 40 is not covered with the hydrogel laminate 20 (swellable gel thin film layer 21).

As illustrated in FIG. 5, in the sensor section 40, each of the unit sensor sections 41 includes a detection surface 43 and a probe 44. The detection surface 43 is an outermost surface (upper surface) of the unit sensor section 41. The detection surface 43 detects a concentration change of an analyte in the vicinity of the sensor section 40. The probe 44 is provided on the detection surface 43 to protrude in the thickness direction of the solid substrate 10. The probe 44 specifically binds to the analyte. Note that the unit sensor section 41 may include one or more types of probes. That is, in the sensor section 40, each of the unit sensor sections 41 may include a probe 45 different from the probe 44 and illustrated in FIG. 6, or may include the probe 44 and the probe 45.

The type and combination of the detection surface 43 of the sensor section 40 and the probe 44 are not limited as long as a target analyte can be detected.

Examples of the material constituting the detection surface 43 include a gold thin film, gold nanoparticles, graphene, and the like.

Examples of the material constituting the probe 44 include an antibody to which an analyte specifically binds, a DNA aptamer whose end is modified with a fluorescent dye, and the like.

In particular, when the detection surface 43 is made of a gold thin film and the probe 44 is made of an antibody, the sensor section 40 is used for surface plasmon measurement. In addition, when the detection surface 43 is made of graphene and the probe 44 is made of a DNA aptamer, the sensor section 40 is used to measure the fluorescence intensity.

### (Sacrificial Layer)

When the sensor section 40 and the swellable gel thin film layer 21 are strongly adhered to each other, and it is difficult to form the hydrogel channel 30 due to buckling and peeling of the swellable gel thin film layer 21 on the non-adhered region 12, a sacrificial layer may be formed in the non-adhered region 12 of the solid substrate 10.

The sacrificial layer is between the solid substrate 10 and the swellable gel thin film layer 21 in at least a part of the non-adhered region 12. A certain region of the sacrificial layer becomes a peeling region that peels off upon application of a predetermined solution stimulation (addition of a chelating agent). The material of the sacrificial layer is not particularly limited as long as the sacrificial layer can be dissolved by a predetermined solution stimulation (addition of a chelating agent). Here, examples of the predetermined solution stimulation include a chelating agent solution stimulation that binds to calcium ions in calcium alginate in an aqueous solution, temperature stimulation, light stimulation, and the like. The sacrificial layer is preferably a thin film capable of maintaining adhesion on one surface 10a of the solid substrate 10 in dry and wet environments (particularly physiological environments).

Examples of the material of the sacrificial layer include biopolymers such as calcium alginate dissolved by addition of calcium chelating agents such as ethylenediaminetetraacetic acid (hereinafter referred to as EDTA), glycol ether diamine tetraacetic acid, 1,2-bis(o-aminophenoxide) ethane-N, N,N′,N′-tetraacetic acid, and citric acid, and dextran decomposable by an enzyme, gelatin exhibiting sol-gel transition by temperature, photoisomerization molecule-containing polymers such as azobenzene and spiropyran exhibiting sol-gel transition by light irradiation, and the like.

The thickness of the sacrificial layer is not particularly limited as long as the hydrogel channel 30 can be formed between the solid substrate 10 and the swellable gel thin film layer 21 after swelling by dissolution stimulation.

### “Hydrogel Laminate”

(Swellable Gel Thin Film Layer) The hydrogel laminate 20 includes a swellable gel thin film layer 21. The swellable gel thin film layer 21 is laminated on one surface 10a of the solid substrate 10 using a hydrogel as a forming material.

Examples of the polymer material constituting the hydrogels include water-soluble polymers such as polyacrylamide and polyvinyl alcohol, polysaccharides such as chitosan and alginic acid, and proteins such as collagen and albumin. These materials have a three-dimensional network structure and swell with a solvent contained in most of the volume. Examples of the solvent in which the polymer material constituting the hydrogel swells include water.

As the polymer material constituting the hydrogel, a stimulus-responsive polymer material can be used. Here, the “stimulus-responsive” property refers to a property in which the polymer material constituting the hydrogel changes its molecular structure in response to a stimulus such as heat, light, electricity, or pH. In the stimulus-responsive hydrogel, the three-dimensional network structure of the polymer material constituting the hydrogel is changed by the stimulus that changes the molecular structure, and the degree of swelling is changed. In the following description, the hydrogel containing the stimulus-responsive polymer material may be referred to as a “stimulus-responsive hydrogel”.

Examples of such a stimulus-responsive polymer material include a polymer material that responds to thermal stimulation, a polymer material that responds pH, a polymer material that responds to light, a polymer material that responds to electrical stimulation, and the like.

Examples of the polymer material that responds to thermal stimulation include poly(N-isopropylacrylamide) and poly(methyl vinyl ether).

Examples of the polymer material that responds to pH include a polymer electrolyte obtained by polymerizing anionic monomers or cationic monomers.

Examples of the polymer material that responds to light include a polymer material having spiropyran or azobenzene in a molecular skeleton.

Examples of the polymer material that responds to electrical stimulation include polypyrrole, polythiophene, and polyaniline.

The material for forming the swellable gel thin film layer 21 may be a hydrogel that responds to multiple stimuli by mixing a plurality of these polymer materials. Furthermore, as the material for forming the swellable gel thin film layer 21, for example, a tough hydrogel such as a double network gel, a slide ring gel, a Tetra-PEG gel, or a nanoclay gel can also be used.

As a method for synthesizing the polymer material constituting the hydrogel, various known methods can be employed. For example, when the polymer material constituting the hydrogel is an acrylic polymer material, the acrylic group may be crosslinked when the acrylic monomer is polymerized to form a three-dimensional network structure.

The type of a polymerization reaction in polymerizing the acrylic monomer is not particularly limited, and examples thereof include radical polymerization using a water-soluble photopolymerization initiator. Examples of the water-soluble photoinitiator include 2-oxoglutaric acid, 4′-(2-hydroxyethoxy)-2-hydroxy-2-methylpropiophenone (trade name: Irgacure 2959), lithium phenyl(2,4,6-trimethylbenzoyl)phosphinate (abbreviation: LAP), 2,2′-azobis [2-methyl-N-(2-hydroxyethyl) propionamide] (trade name: VA-086), and the like.

At the time of radical polymerization, a deoxidizing agent may be added to a reaction system in order to prevent polymerization inhibition by oxygen. Examples of the deoxidizing agent include a combination of glucose and glucose oxidase. Further, the radical polymerization may be performed under an inert gas atmosphere such as nitrogen or argon.

When the polymer material constituting the hydrogel is a polysaccharide or a protein, a three-dimensional network structure may be formed by physical bonding of the polysaccharide or the protein, or the polysaccharide or the protein may be crosslinked using a crosslinking agent to form a three-dimensional network structure. Examples of the crosslinking agent include glutaraldehyde.

The shape of the swellable gel thin film layer 21 is not particularly limited, and various shapes can be selected according to a use form. For example, the swellable gel thin film layer 21 may have a film shape, a plate shape, a block shape, or the like. Among them, the shape of the swellable gel thin film layer 21 is preferably a film shape.

The thickness of the swellable gel thin film layer 21 is not particularly limited, but is preferably a thickness that exhibits structural strength to the extent that it is not crushed by its own weight. For example, when a hydrogel containing polyacrylamide is used as a material for forming the swellable gel thin film layer 21, the thickness of the swellable gel thin film layer 21 is preferably 50 μm to 1000 μm, and more preferably 120 μm to 200 μm.

The strength of the swellable gel thin film layer 21 can be improved by increasing the crosslinking of the polymer material constituting the hydrogel through chemical crosslinking or physical crosslinking, or increasing the concentration of the polymer material constituting the hydrogel.

For example, when a hydrogel containing polyacrylamide is prepared by polymerizing a monomer (precursor) of acrylamide, the monomer concentration is preferably 0.8 mol/L to 8 mol/L, and more preferably 2 mol/L to 4 mol/L.

When methylene bisacrylamide is used as a chemical crosslinking agent in the case of polymerizing a monomer of acrylamide, the crosslinking agent concentration is preferably 0.01 mol % to 20 mol %, and more preferably 0.03 mol % to 1 mol % with respect to the monomer.

The hydrogel can contain various additives. The type of the additive is not particularly limited as long as the additive does not inhibit hydrogel formation. Examples of the additive include a biomolecule for improving bioaffinity, a silver nanoparticle or a surfactant for exhibiting antibacterial properties, an ionic liquid or a conductive polymer for increasing conductivity, a magnetic nanoparticle for reacting with a magnetic field, and the like. By adding these additives to the hydrogel, an arbitrary function can be imparted to the hydrogel.

### (Non-Swellable Gel Thin Film Layer)

The hydrogel laminate 20 includes a non-swellable gel layer 22 on the swellable gel thin film layer 21. The non-swellable gel layer 22 is laminated on the swellable gel thin film layer 21 using a hydrogel as a forming material.

The non-swellable gel layer 22 is a gel after the polymer material constituting the hydrogel swells. That is, the non-swellable gel layer 22 is swollen by a liquid such as water flowing into a network structure or the like of a polymer material. Therefore, it can also be said that the non-swellable gel layer 22 is a swollen product of a polymer material.

The polymer material constituting the non-swellable gel layer 22 has a lower degree of swelling than the polymer material constituting the swellable gel thin film layer 21. The degree of swelling of the polymer material constituting the non-swellable gel layer 22 is not particularly limited as long as it is lower than the degree of swelling of the polymer material constituting the swellable gel thin film layer 21. The degree of swelling of the polymer material constituting the non-swellable gel layer 22 is desirably, for example, about 0.8 times to 1.2 times, based on the size before swelling in one direction.

Here, the “degree of swelling” can be calculated by the following formula (1), for example, by cutting out a polymer material constituting the swellable gel thin film layer 21 immediately after polymerization or a polymer material constituting the non-swellable gel layer 22 as a disk-shaped sample having an appropriate diameter, and leaving the disk-shaped sample in pure water until no change in size occurs.

(Degree of swelling)=D/D₀ (1)

In the above formula (1), D is the diameter of the largest portion of the sample after being left still in pure water, and D₀ is the diameter of the circular sample before being left still in pure water.

The polymer material constituting the non-swellable gel layer 22 may be a hydrogel or a gel other than the hydrogel. When the polymer material constituting the non-swellable gel layer 22 is a hydrogel, the polymer material constituting the non-swellable gel layer 22 may be the same as or different from the polymer material constituting the swellable gel thin film layer 21.

Examples of the polymer material constituting the non-swellable gel layer 22 include a chemically crosslinked gel crosslinked by a covalent bond from a radical polymerization reaction of monomers. Examples of the chemically crosslinked gel include polyacrylamide and derivatives thereof (polydimethylacrylamide, poly N-isopropylacrylamide, etc.). In this case, by using methylene bisacrylamide as the crosslinking agent, the crosslinking density may be increased, and the degree of swelling of the polymer material constituting the non-swellable gel layer 22 may fall within the above numerical range.

Examples of the polymer material constituting the non-swellable gel layer 22 also include a physically crosslinked gel in which a polymer having a positive charge or a negative charge and an ion having a polyvalent charge opposite thereto are combined.

Examples of the physically crosslinked gel include a physically crosslinked gel gelled by combining a sodium alginate solution, which is a polymer having a negative charge, and a calcium solution such as calcium chloride or calcium sulfate. Other examples include physically crosslinked gels in which poly(2,2′-disulfo-4,4′-bensidineterephthalamide: PBDT), which is a water-soluble polyaramid, and various metal polyvalent cations (Ca²⁺, Fe²⁺, Al³⁺, Zr⁴⁺, Ti⁴⁺, etc.) are combined. Instead of PBDT, TEMPO-oxidized cellulose nanofibers (NIPPON PAPER INDUSTRIES CO., LTD.) that are also negatively charged, and cellulose nanofibers (Oji Holdings Corporation) defibrated by a phosphoric acid esterification method may be used.

Here, “TEMPO” is an abbreviation for 2,2,6,6-tetramethylpiperidine 1-oxyl (radical).

The non-swellable gel layer 22 covers one surface of the swellable gel thin film layer 21 outside the hydrogel channel 30. The non-swellable gel layer 22 covers the surface on which the swellable gel thin film layer 21 is not in contact with the solid substrate 10. That is, one surface of the swellable gel thin film layer 21 covered with the non-swellable gel layer 22 is one surface opposite to the surface in contact with the solid substrate 10 (that is, the surface facing the surface in contact with the solid substrate 10).

The non-swellable gel layer 22 covers the outside of the hydrogel channel 30. Therefore, when an aqueous liquid is poured inside the hydrogel channel 30, the aqueous liquid permeates the swellable gel thin film layer 21, diffuses to the outside of the hydrogel channel 30, and reaches the non-swellable gel layer 22.

For example, when the non-swellable gel layer 22 is composed of a hydrogel, the aqueous liquid that has reached the non-swellable gel layer 22 can diffuse into the non-swellable gel layer 22. Therefore, by disposing an arbitrary object (for example, cells, cultured tissues) in advance inside the non-swellable gel layer 22, the aqueous liquid can be selectively supplied to the object in a predetermined region inside the non-swellable gel layer 22.

For example, when a hydrogel having a positive charge or a negative charge is used as a polymer material constituting the non-swellable gel layer 22, it is possible to provide the hydrogel channel 30 with a function of preventing diffusion of low molecules having a specific charge. That is, the non-swellable gel layer 22 can be provided with a function of blocking low molecules having a specific charge from diffusing from the inside to the outside of the hydrogel channel 30.

In addition, for example, as a polymer material constituting the non-swellable gel layer 22, a hydrogel in which hydrophilic and hydrophobic properties are switched in response to an external stimulus; and a hydrogel whose degree of swelling can be changed in response to an external stimulus can also be used.

In the case of using a hydrogel in which hydrophilic and hydrophobic properties are switched in response to an external stimulus, when the non-swellable gel layer 22 is hydrophilic as a result of responding to an external stimulus, the low molecules diffusing in the non-swellable gel layer 22 can be selectively limited to those that are hydrophilic. On the other hand, when the non-swellable gel layer 22 is hydrophobic as a result of responding to an external stimulus, the low molecules diffusing in the non-swellable gel layer 22 can be selectively limited to those that are hydrophobic.

In the case of using a hydrogel capable of changing the swelling rate (water content) in response to an external stimulus, when the swelling rate of the non-swellable gel layer 22 is relatively high as a result of responding to an external stimulus, the diffusion rate of low molecules diffusing in the non-swellable gel layer 22 becomes relatively slow.

On the other hand, when the swelling rate of the non-swellable gel layer 22 is relatively low as a result of responding to an external stimulus, the diffusion rate of low molecules diffusing in the non-swellable gel layer 22 becomes relatively high.

In addition, the non-swellable gel layer 22 may have a functional group exhibiting a predetermined response such as fluorescence to low molecules diffused from the hydrogel channel 30. In this case, when the low molecules diffuse in the non-swellable gel layer 22, the non-swellable gel layer 22 exhibits a predetermined response such as fluorescence, and therefore a function as a sensor of the diffused low molecules can be imparted to the hydrogel channel type device 1 with a sensor.

The mechanical strength of the non-swellable gel layer 22 is not particularly limited. For example, when the elastic modulus (to 1.3 MPa) equivalent to that of polydimethylsiloxane (PDMS) is obtained in the non-swellable gel layer 22, a double network gel in which a physically crosslinked gel and a chemically crosslinked gel are combined is preferable as a polymer material constituting the non-swellable gel layer 22. Since the double network gel has a tough double network structure, the mechanical strength is further improved.

The shape of the non-swellable gel layer 22 is not particularly limited. However, the thickness of the non-swellable gel layer 22 needs to be thicker than the height of the hydrogel channel 30 in order to cover the hydrogel channel 30. The thickness of the non-swellable gel layer 22 may be further increased in order to give sufficient strength to the joint portion between the liquid feeding tube 50 and the hydrogel laminate 20.

The polymer material constituting the non-swellable gel layer 22 may contain various additives as long as the degree of swelling does not change extremely. By using an arbitrary additive, the non-swellable gel layer 22 can be given an arbitrary function.

The additive in the non-swellable gel layer 22 is not particularly limited as long as it does not inhibit gel formation. For example, a biomolecule for improving bioaffinity; a silver nanoparticle or a surfactant for exhibiting antibacterial properties; an ionic liquid or a conductive polymer for increasing conductivity; a magnetic nanoparticle for reacting with a magnetic field; a protein that binds to glucose to enhance fluorescence intensity, and the like can be mentioned.

The method for synthesizing the polymer material constituting the non-swellable gel layer 22 is not particularly limited as long as it is a method in which the degree of swelling is lower than that of the polymer material constituting the swellable gel thin film layer 21.

### (Hydrogel Channel)

The hydrogel channel 30 is formed between the solid substrate 10 and the hydrogel laminate 20. The hydrogel channel 30 is formed by separating the polymer material constituting the swellable gel thin film layer 21 on the non-adhered region 12 by swelling of the polymer material constituting the swellable gel thin film layer 21 at the non-adhered region 12 of the solid substrate 10.

Specifically, at the interface between the solid substrate 10 and the swellable gel thin film layer 21, the position of the portion where the polymer material constituting the swellable gel thin film layer 21 is separated from the solid substrate 10 is controlled by the pattern arrangement of the adhered region 11 and the non-adhered region 12. When the polymer material constituting the swellable gel thin film layer 21 swells and the polymer material constituting the swellable gel thin film layer 21 on the non-adhered region 12 is selectively separated in the non-adhered region 12 of the solid substrate 10, buckling deformation of the polymer material constituting the swellable gel thin film layer 21 occurs. As a result, the hybrid channel, that is, the hydrogel channel 30, is formed as a space surrounded by the solid substrate 10 and the swellable gel thin film layer 21.

The hydrogel channel 30 includes the swellable gel thin film layer 21 at a portion separated from the solid substrate 10 as a channel surface 30c. The hydrogel channel 30 has a first open end surface 30a and a second open end surface 30b. The channel surface 30c of the hydrogel channel 30 is formed in a band shape along the extending direction of the non-adhered region 12 between the first open end surface 30a and the second open end surface 30b. The hydrogel channel 30 formed at the interface between the solid substrate 10 and the swellable gel thin film layer 21 penetrates the non-swellable gel layer 22 from a first end surface 22a side to a second end surface 22b side.

### “Liquid Feeding Tube”

The liquid feeding tube 50 is fixed to the first open end surface 30a and the second open end surface 30b of the hydrogel channel 30 with an adhesive. Specifically, in each of the first open end surface 30a and the second open end surface 30b of the hydrogel channel 30, the liquid feeding tube 50 is fixed between the solid substrate 10 and the hydrogel laminate 20 with an adhesive.

The liquid feeding tube 50 is a tube for supplying an arbitrary fluid into the hydrogel channel 30.

The liquid feeding tube 50 is not particularly limited as long as the tube has a form capable of feeding a liquid from an outside. The type of the liquid feeding tube 50 is not particularly limited. Examples of the liquid feeding tube 50 include tubes made of polytetrafluoroethylene (PTFE), tetrafluoroethylene (PFA), polyurethane, polyethylene, silicone, polyimide, and the like. The outer diameter of the liquid feeding tube 50 is not particularly limited. Here, the outer diameter of the liquid feeding tube 50 is desirably about the same as the height of the hydrogel channel 30.

The adhesive fixes the liquid feeding tube 50 to the hydrogel channel 30. That is, the adhesive fixes the liquid feeding tube 50 between the solid substrate 10 and the hydrogel laminate 20.

In the hydrogel channel type device 1 with a sensor, at the first open end surface 30a and the second open end surface 30b of the hydrogel channel 30, the periphery of the hydrogel channel 30 is densely filled with the adhesive in the space in contact with the channel surface 30c of the hydrogel channel 30. The adhesive desirably has water resistance and adhesiveness with solid substrate 10 and hydrogel laminate 20. Examples of the adhesive include a cyanoacrylate-based adhesive, a silicone-based adhesive, an epoxy-based adhesive, and the like.

### “Mechanism of Action”

In the hydrogel channel type device 1 with a sensor described above, when a tissue-like structure including epithelial cells is formed on an upper surface 20a of the hydrogel laminate 20 (an upper surface 22c of the non-swellable gel layer 22), the hydrogel channel 30 formed between the solid substrate 10 and the hydrogel laminate 20 is regarded as a circulatory system tubular tissue such as a blood vessel and a lymph, and the non-swellable gel layer 22 is regarded as an interstitial tissue, and therefore the structure of the skin and the structure of a digestive organ such as an esophagus and an intestine can be simulated.

In the hydrogel channel type device 1 with a sensor described above, vascular endothelial cells can be cultured on the inner wall of the hydrogel channel 30 formed between the solid substrate 10 and the hydrogel laminate 20, fibroblasts can be cultured inside the non-swellable gel layer 22, and epithelial cells can be cultured on the upper surface 20a of the hydrogel laminate 20. Therefore, it is also possible to create an MPS that has a composition closer to that of a living tissue.

In the hydrogel channel type device 1 with a sensor described above, the network size and hardness of the swellable gel thin film layer 21 and the non-swellable gel layer 22 can be adjusted by the hydrogel composition, and by controlling the physical properties of the hydrogel, it can be used as a diseased tissue model of a living tissue. For example, it is possible to simulate local changes in physical properties inside a living tissue due to fibrosis, scarring, or the like of the living tissue.

In the hydrogel channel type device 1 with a sensor described above, the sensor section 40 can include one or more types of probes on the detection surface of the unit sensor section 41. Therefore, the dynamics of a plurality of analytes can be evaluated.

In the hydrogel channel type device 1 with a sensor described above, the sensor section 40 can include one or more types of probes on the detection surface of the unit sensor section 41. Therefore, by simultaneously providing a probe using an optical detection method and a probe using an electrochemical detection method, a detection method suitable for an analyte can be selected.

In the hydrogel channel type device 1 with a sensor described above, since the sensor section 40 is provided on the solid substrate 10, information that can be acquired by the sensor section 40 is only two-dimensional information, but it is possible to estimate the spatial distribution in the diffusion process of the analyte by assuming that the diffusion of the analyte inside the hydrogel laminate 20 is isotropic. For example, as illustrated in FIG. 1, when the height from the inner wall of the hydrogel channel 30 to the upper surface 20a of the hydrogel laminate 20 is d, the arrival time of the analyte on the upper surface 20a of the hydrogel laminate 20 can be estimated by detecting the signal of the analyte on the solid substrate 10 at a distance d from the inner wall of the hydrogel channel 30.

“Method for Using Hydrogel Channel Type Device with Sensor”
<Detection of Diffusion of Analyte Solution Using Hydrogel Channel Type Device with Surface Plasmon Resonance (SPR) Sensor>

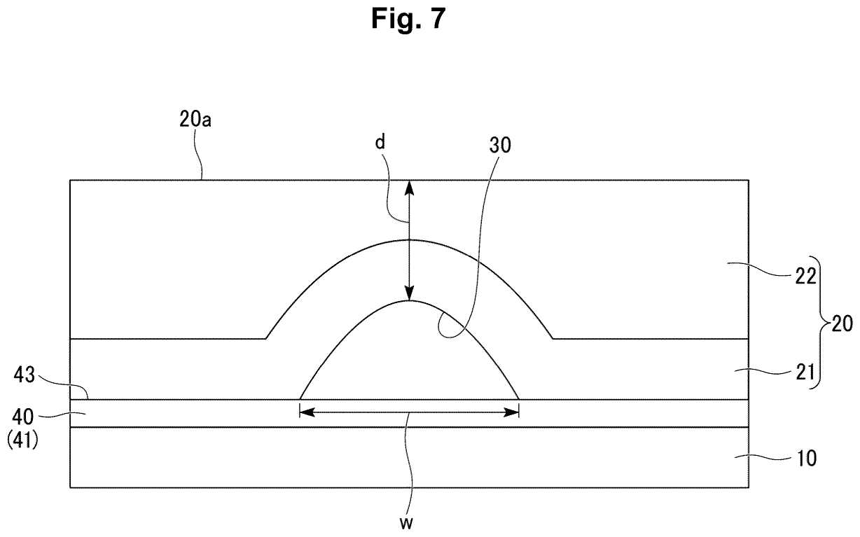

FIGS. 7 to 10 are side views illustrating the concept of diffusion of an analyte solution by a hydrogel channel type device with an SPR sensor and detection by the SPR sensor.

As illustrated in FIG. 7, for example, a gold thin film sputtered on one surface 10a of the solid substrate 10 is used as the detection surface 43 of the sensor section 40. A size w of the detection surface 43 in the radial direction of the hydrogel channel 30 is preferably wider than a height d from the inner wall of the hydrogel channel 30 to the upper surface 20a of the hydrogel laminate 20. FIG. 7 illustrates a state before an analyte solution 100 is fed into the hydrogel channel 30. In addition, to in FIG. 7 indicates that it is before the analyte solution 100 is fed into the hydrogel channel 30.

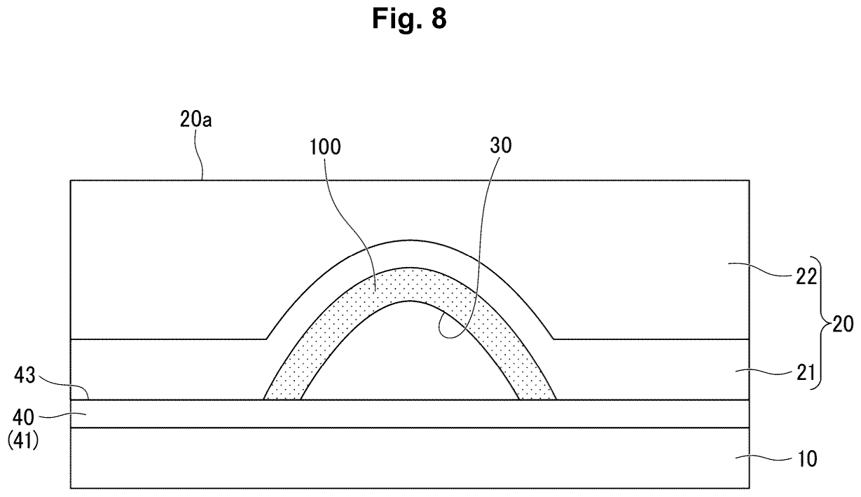

As illustrated in FIG. 8, when the analyte solution 100 is fed into the hydrogel channel 30, the analyte solution 100 diffuses into the swellable gel thin film layer 21. In addition, t₁ in FIG. 8 indicates that it is immediately after the analyte solution 100 is fed into the hydrogel channel 30.

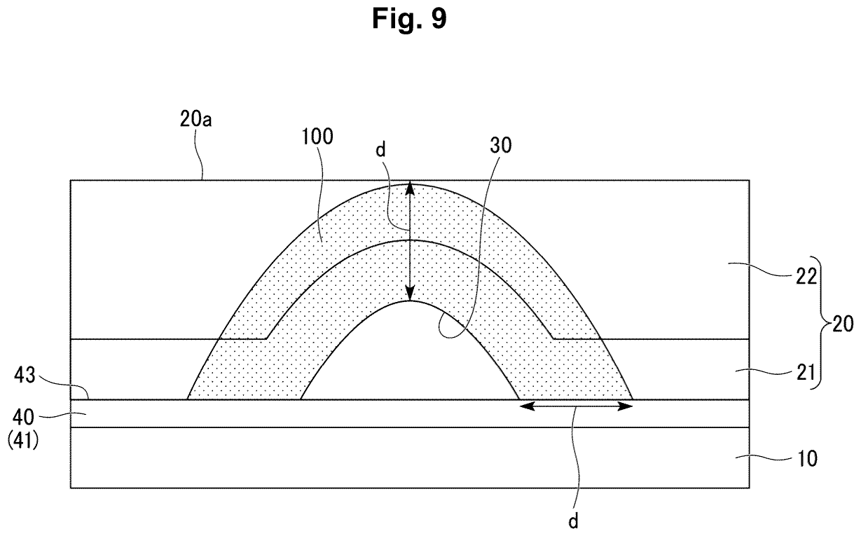

In addition, as time passes, the analyte solution 100 also diffuses into the non-swellable gel layer 22 as illustrated in FIG. 9, and the analyte solution 100 reaches the upper surface 20a of the hydrogel laminate 20. In addition, t₂ in FIG. 9 indicates that a certain time has elapsed since the analyte solution 100 was fed into the hydrogel channel 30.

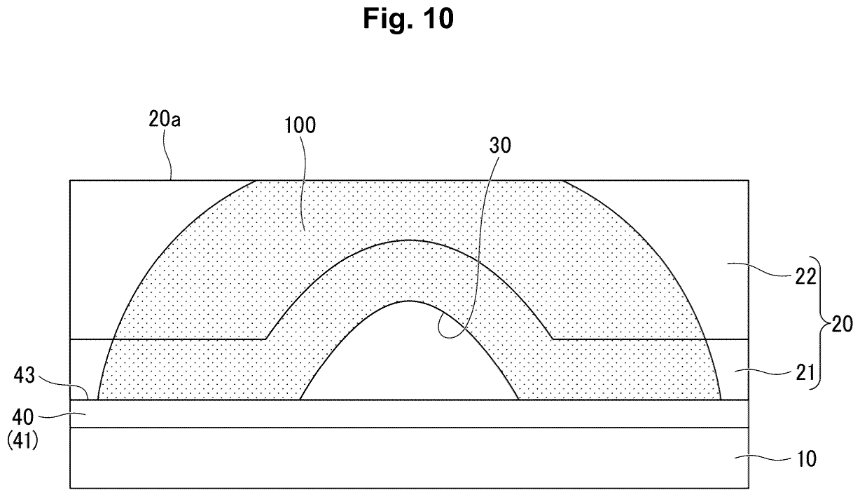

Further, as time passes, as illustrated in FIG. 10, the analyte solution 100 diffuses over a wide range of the non-swellable gel layer 22. In addition, t₃ in FIG. 10 indicates that a considerable amount of time has elapsed since the analyte solution 100 was fed into the hydrogel channel 30.

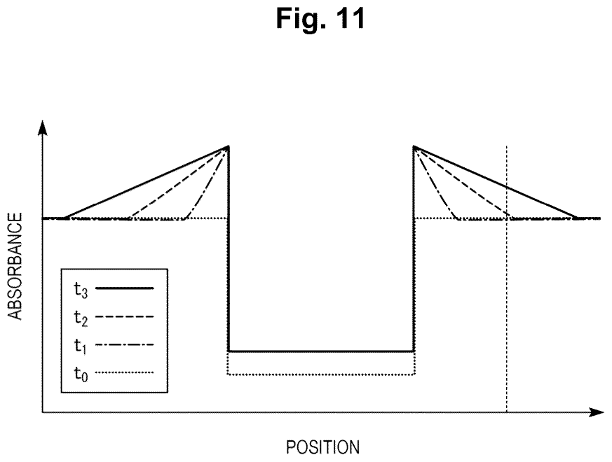

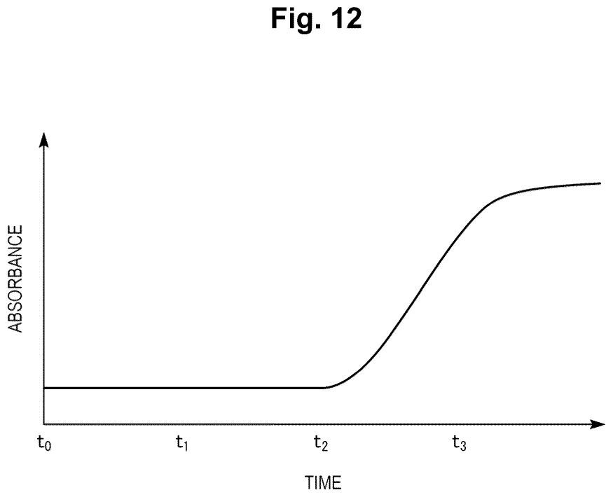

The diffusion of the analyte solution 100 is detected by measuring a change in an absorption spectrum of light on the detection surface 43 of the sensor section 40 using the SPR sensor. As a result, as illustrated in FIG. 11, the relationship between the diffusion position of the analyte solution 100 with respect to the hydrogel laminate 20 and the diffusion time (elapsed time after the analyte solution 100 is fed into the hydrogel channel 30, and time when the analyte solution 100 reaches the upper surface 20a of the hydrogel laminate 20) of the analyte solution 100 with respect to the hydrogel laminate 20 is obtained. In addition, as illustrated in FIG. 12, the relationship between the diffusion time (elapsed time after the analyte solution 100 is fed into the hydrogel channel 30, and time when the analyte solution 100 reaches the upper surface 20a of the hydrogel laminate 20) of the analyte solution 100 with respect to the hydrogel laminate 20 and the result of measuring the change in the absorption spectrum of light on the detection surface 43 of the sensor section 40 (the intensity of the absorption spectrum) is obtained.

The sensor section 40 may include a probe on the detection surface 43. The sensor section 40 may include one type of probe on the detection surface 43, or may include two or more types of probes provided in an array.

The probe is not particularly limited, and examples thereof include an antibody, a DNA aptamer, and the like which are easily immobilized on the surface of the gold thin film.

<Detection of Diffusion of Analyte Solution Using Hydrogel Channel Type Device with Graphene/DNA Aptamer Sensor>

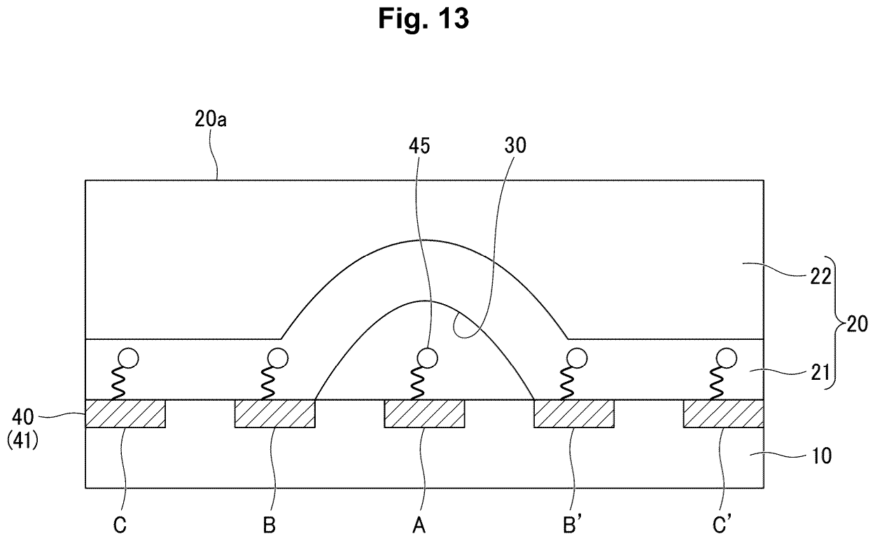

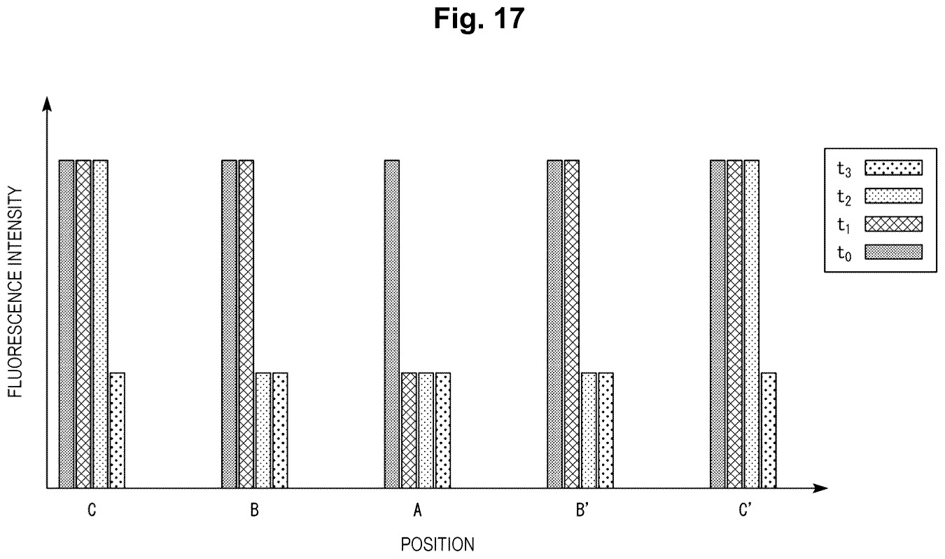

FIGS. 13 to 16 are side views illustrating the concept of diffusion of an analyte solution by a hydrogel channel type device with a graphene/DNA aptamer sensor and detection by the graphene/DNA aptamer sensor. In FIGS. 13 to 16, the case where the fluorescence intensity of the fluorescent dye at the end of the DNA aptamer is high is indicated by a white circle, and the case where the fluorescence intensity of the fluorescent dye at the end of the DNA aptamer is low is indicated by a black circle. Furthermore, in FIGS. 13 to 16, the sensor sections 40 indicated by A, B, B′, C, and C′ correspond to A, B, B′, C, and C′ illustrated in FIG. 17.

As illustrated in FIG. 13, for example, graphene or graphene oxide immobilized on one surface 10a of the solid substrate 10 is used as the detection surface 43 of the sensor section 40. The method for immobilizing graphene or graphene oxide on one surface 10a of the solid substrate 10 is not particularly limited, and examples thereof include a method in which photolithography and oxygen plasma etching are combined, an inkjet printing method, and the like. The sensor section 40 includes a probe 45 on the detection surface 43. Examples of the probe 45 include DNA aptamers. The DNA aptamer preferably has a fluorescent dye at the end that is not fixed to the detection surface 43, and has a functional group that binds to the detection surface 43 modified at the end that is fixed to the detection surface 43. The fluorescent dye at the end of the DNA aptamer is not particularly limited as long as the structure is changed by binding to the analyte, and the fluorescent dye is located in the vicinity of the detection surface 43 and causes fluorescence quenching due to light energy transfer. Examples of such a fluorescent dye include fluorescein and rhodamine. The functional group for immobilizing the DNA aptamer on the detection surface 43 is not particularly limited as long as binding can be maintained under physiological conditions, and examples thereof include pyrene and an amino group. FIG. 13 illustrates a state before the analyte solution 100 is fed into the hydrogel channel 30. In addition, to in FIG. 13 indicates that it is before the analyte solution 100 is fed into the hydrogel channel 30.

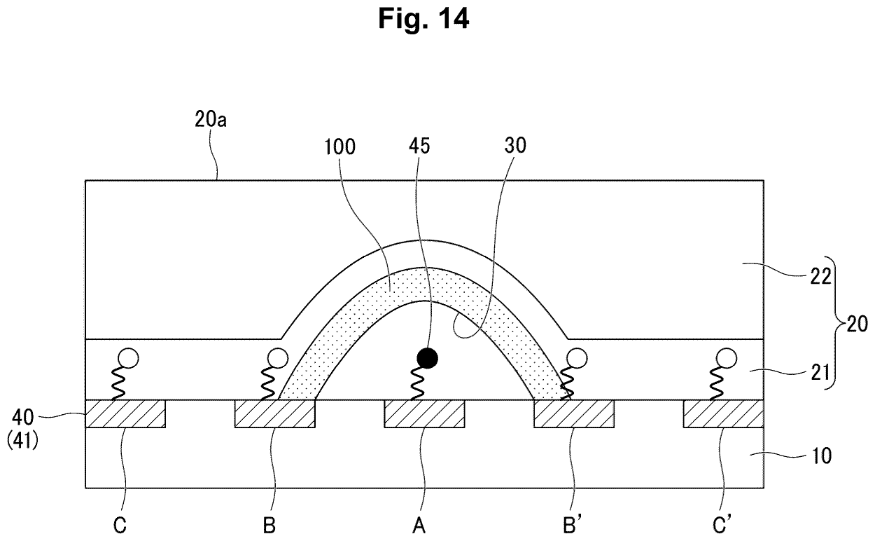

As illustrated in FIG. 14, when the analyte solution 100 is fed into the hydrogel channel 30, the analyte solution 100 diffuses into the swellable gel thin film layer 21. In addition, t₁ in FIG. 14 indicates that it is immediately after the analyte solution is fed into the hydrogel channel 30.

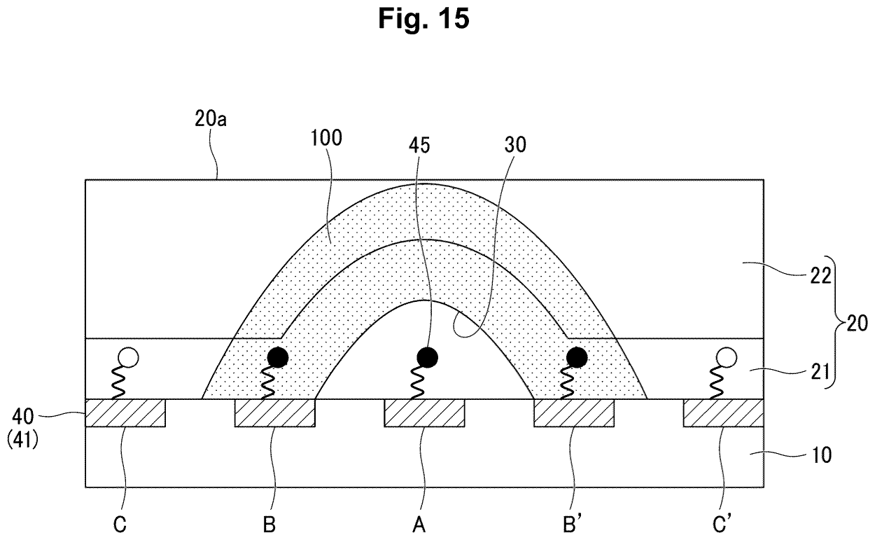

In addition, as time passes, the analyte solution 100 also diffuses into the non-swellable gel layer 22 as illustrated in FIG. 15, and the analyte solution 100 reaches the upper surface 20a of the hydrogel laminate 20. In addition, t₂ in FIG. 15 indicates that a certain time has elapsed since the analyte solution was fed into the hydrogel channel 30.

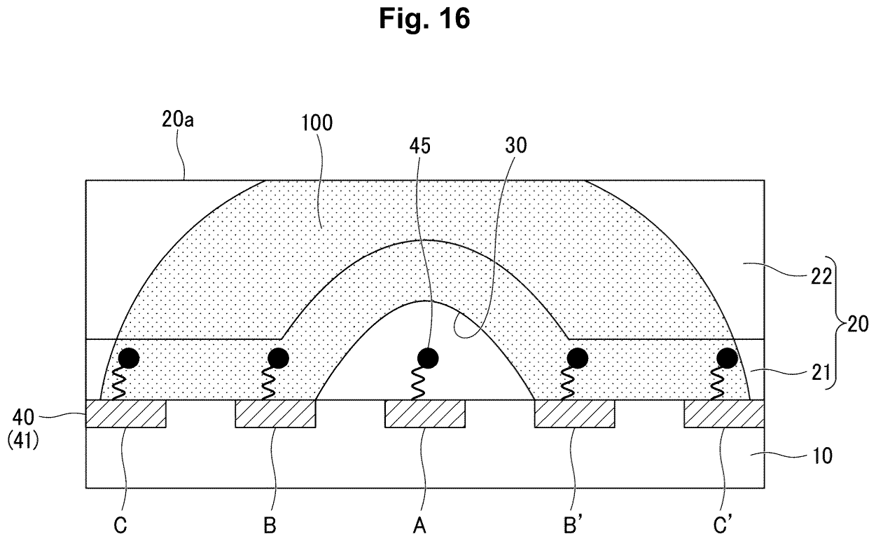

Further, as time passes, as illustrated in FIG. 16, the analyte solution 100 diffuses over a wide range of the non-swellable gel layer 22. In addition, t₃ in FIG. 16 indicates that a considerable amount of time has elapsed since the analyte solution was fed into the hydrogel channel 30.

Diffusion of the analyte solution 100 is detected by measuring fluorescence quenching (fluorescence intensity) of a fluorescent dye at the end of the DNA aptamer with a graphene/DNA aptamer sensor. As a result, as illustrated in FIG. 17, the relationship between the diffusion position of the analyte solution 100 with respect to the hydrogel laminate 20 and the fluorescence quenching (fluorescence intensity) of the fluorescent dye at the end of the DNA aptamer is obtained. When the analyte solution 100 comes into contact with the fluorescent dye at the end of the DNA aptamer, the fluorescence intensity of the fluorescent dye becomes weak, and it can be detected that the analyte solution 100 has diffused to the position of the corresponding DNA aptamer.

[Method for Manufacturing Hydrogel Channel Type Device with Sensor]

In an example of a method for manufacturing a hydrogel channel type device with a sensor according to an embodiment of the present invention, as illustrated in FIGS. 2 and 3, the adhered region 11 and the non-adhered region 12 are formed on one surface 10a of the solid substrate 10 provided with the sensor section 40. The method for forming the adhered region 11 and the non-adhered region 12 is not particularly limited, and examples thereof include a method using photolithography using a positive photoresist and oxygen plasma etching, and a stencil method in which negative types of the adhered region 11 and the non-adhered region 12 are prepared and oxygen plasma etching is performed.

Next, a polymer material constituting the swellable gel thin film layer 21, in other words, a hydrogel precursor solution is added dropwise to one surface 10a of the solid substrate 10, and arbitrary radical polymerization is performed to form the swellable gel thin film layer 21. The method for adhering the swellable gel thin film layer 21 and the solid substrate 10 to each other is not particularly limited, and examples thereof include a method for adhering one surface 10a of the solid substrate 10 and the swellable gel thin film layer 21 to each other by a covalent bond. For example, one surface 10a of the solid substrate 10 is modified with 3-(methacryloyloxy) propyltrimethoxysilane (TMSPMA), a hydrogel precursor solution gelled by radical polymerization is added dropwise thereon, and arbitrary radical polymerization is performed. When a gold thin film is used for the detection surface 43 of the sensor section 40 provided on one surface 10a of the solid substrate 10, the swellable gel thin film layer 21 and the solid substrate 10 may be adhered to each other by modifying the surface of the gold thin film with a compound having a dithiol such as bis(2-methacryloyl)oxyethyl disulfide (Bis-thiol) and an acrylic group.

Alternatively, a porous thin film may be deposited on one surface 10a of the solid substrate 10, and the swellable gel thin film layer 21 and the solid substrate 10 may be adhered to each other by mutual invagination.

Next, a polymer material constituting the non-swellable gel layer 22, in other words, a hydrogel precursor solution is added dropwise onto the swellable gel thin film layer 21, and arbitrary radical polymerization is performed to form the non-swellable gel layer 22.

Next, the polymer material constituting the swellable gel thin film layer 21 is swollen (gelled) to selectively separate the polymer material constituting the swellable gel thin film layer 21 on the non-adhered region 12 from the solid substrate 10, thereby causing buckling deformation of the polymer material constituting the swellable gel thin film layer 21 to form the hydrogel channel 30 as a space surrounded by the solid substrate 10 and the swellable gel thin film layer 21.

Next, the liquid feeding tube 50 is inserted into both ends of the hydrogel channel 30, and the liquid feeding tube 50 is adhered and fixed between the solid substrate 10 and the hydrogel laminate 20 with an adhesive to obtain the hydrogel channel type device 1 with a sensor.

### Other Embodiments

Note that the present invention is not limited to the above-described embodiments.

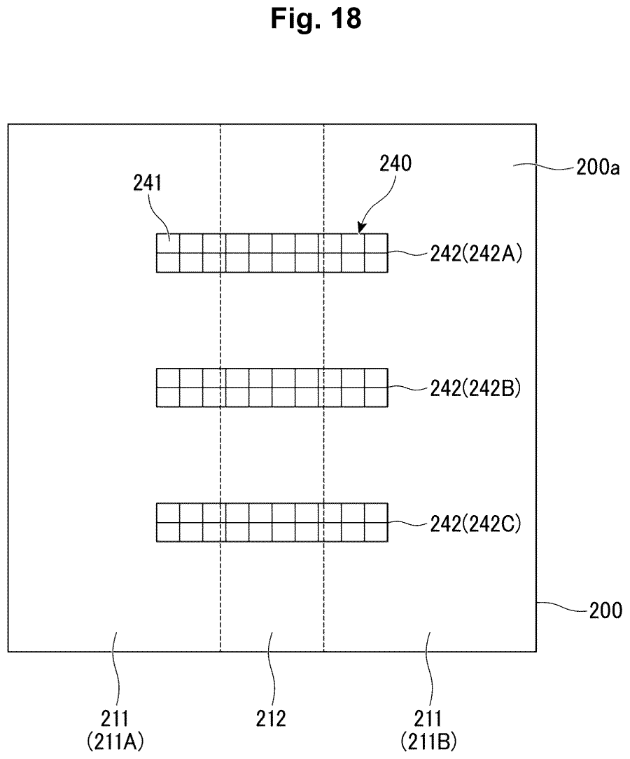

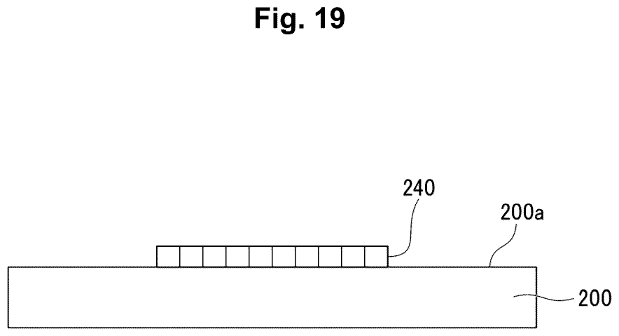

For example, a modification example as illustrated in FIGS. 18 and 19 may be employed.

### Modification Example

Similarly to the solid substrate 10 described above, a solid substrate 200 of a modification example illustrated in FIGS. 18 and 19 has adhered regions 211 that are adhered to the hydrogel laminate 20 and a non-adhered region 212 that is not adhered to the hydrogel laminate 20 at the interface with the hydrogel laminate 20. The adhered region 211 has a first adhered region 211A and a second adhered region 211B with the non-adhered region 212 in between.

The first adhered region 211A and the second adhered region 211B have the same configurations as those of the adhered region 11 and the non-adhered region 12 described above.

A sensor section 240 is provided at an interface between the solid substrate 200 and the hydrogel laminate 20, that is, on one surface 200a of the solid substrate 200.

The sensor section 240 is provided on the adhered region 211 and the non-adhered region 212. The sensor section 240 includes a plurality of unit sensor section rows 242 each including two or more unit sensor sections 241 arranged adjacent to each other in the width direction of the adhered region 211 and the non-adhered region 212. Here, a case where the sensor section 240 includes a first unit sensor section row 242A, a second unit sensor section row 242B, and a third unit sensor section row 242C each including the unit sensor sections 241 will be exemplified. In addition, the first unit sensor section row 242A, the second unit sensor section row 242B, and the third unit sensor section row 242C are arranged apart from each other in the length direction of the adhered region 211 and the non-adhered region 212.

### Examples

Hereinafter, the present invention will be described more specifically with reference to Examples, but the present invention is not limited to the following Examples.

### Examples

<Detection of Diffusion of Analyte Solution Using Hydrogel Channel Type Device with Sensor in which Gold Thin Film is Used as Detection Surface of Sensor Section>

As a solid substrate with a sensor in which a gold thin film is used as a detection surface of a sensor section (hereinafter referred to as a “glass substrate with a gold thin film”), one obtained by forming a gold thin film by sputtering gold at the center of one surface of a slide glass was used.

The glass substrate with a gold thin film was cleaned by oxygen plasma treatment.

Thereafter, a sodium alginate solution was added dropwise to the surface of the glass substrate with a gold thin film on which the gold thin film was formed. Subsequently, the surface of the glass substrate with a gold thin film on which the gold thin film was formed was spin-coated with a sodium alginate solution to obtain a glass substrate with a spin-coated gold thin film.

The glass substrate with a spin-coated gold thin film was immersed in an aqueous solution of calcium chloride, and then the glass substrate with a spin-coated gold thin film was washed with ultrapure water and then dried to form a calcium alginate thin film as a sacrificial layer, thereby obtaining a glass substrate with a calcium alginate thin film.

A PMMA thin film and a positive photoresist thin film were sequentially laminated on one surface of the glass substrate with a calcium alginate thin film by spin coating.

Next, the positive photoresist thin film was formed into a channel shape by UV exposure through a photomask and development processing.

Next, the solid substrate from which the PMMA thin film and the calcium alginate thin film had been removed by oxygen plasma etching was immersed in a toluene solution containing 25 mmol/L of TMSPMA and 25 mmol/L of Bis-thiol to form an adhered region on one surface of the solid substrate.

Next, the solid substrate was immersed in acetone to remove the PMMA thin film and the positive photoresist thin film, thereby obtaining a solid substrate having one surface composed of a sacrificial layer and an adhered region.

Next, spacers having a thickness of 80 μm were disposed on both end surfaces of the solid substrate with a sacrificial layer obtained by the above-described method, and an aqueous solution containing acrylamide, methylene bisacrylamide, and LAP was added dropwise onto the solid substrate with a sacrificial layer as a precursor solution of a swellable film-shaped gel.

One surface of the solid substrate with a sacrificial layer was covered with a cover glass, and irradiated with light having a wavelength of 365 nm to gelate the precursor solution, thereby forming a swellable gel thin film layer.

After the swellable gel thin film layer was formed, the cover glass covering one surface of the solid substrate with a sacrificial layer was removed, and unreacted gel precursor molecules were removed in pure water.

Next, the solid substrate with a sacrificial layer was immersed in a 10 mmol/L EDTA aqueous solution that serves as a dissolution stimulus, so that the sacrificial layer composed of the calcium alginate thin film was dissolved, and the swellable gel thin film layer on the upper surface of the sacrificial layer was peeled off from the solid substrate and swollen to form a hydrogel channel.

Next, a liquid feeding tube was inserted into both ends of the hydrogel channel, and the liquid feeding tube was adhered and fixed between the swellable gel thin film and the solid substrate with an adhesive.

A rhodamine solution was fed into the hydrogel channel from the liquid feeding tube, and the time change of the SPR signal intensity in the vicinity of the inner wall of the hydrogel channel was measured.

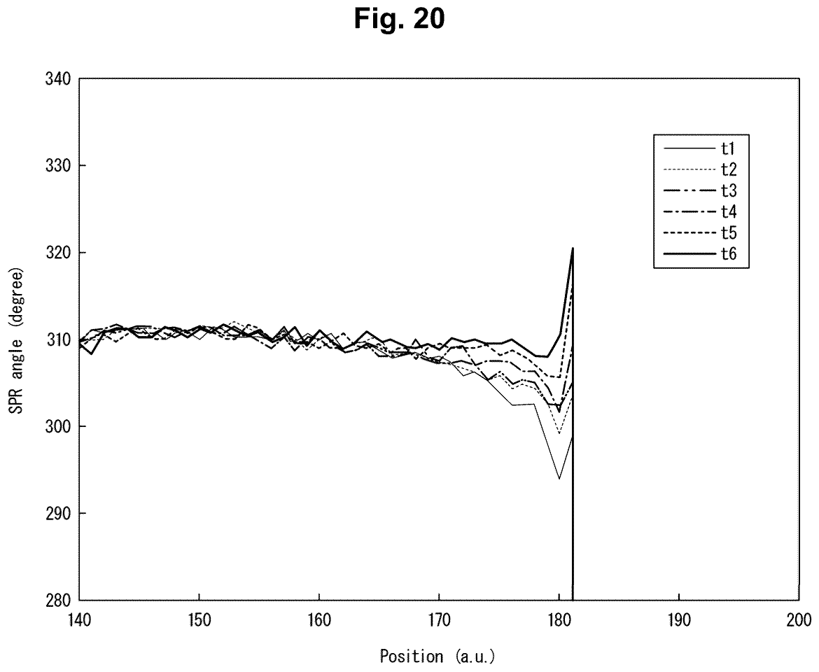

FIG. 20 illustrates the results of measuring the diffusion of a rhodamine solution in a hydrogel channel type device with an SPR sensor using the SPR sensor.

From the results illustrated in FIG. 20, it can be confirmed that the fluorescence intensity derived from rhodamine inside the hydrogel laminate increases as time passes time t₁ to time to.

Although the embodiments of the present invention have been described in detail with reference to the drawings, specific configurations are not limited to the embodiments, and include design and the like within the scope of the present invention without departing from the gist of the present invention.

### INDUSTRIAL APPLICABILITY

The hydrogel channel type device with a sensor according to the present invention is useful as a cell culture device, a microreactor, and a sensing device utilizing a diffusible channel shape, and is widely applicable to pharmacology, tissue engineering, chemical engineering, and the like.

### REFERENCE SIGNS LIST

1 Hydrogel channel type device with sensor
10 Solid substrate
11 Adhered region
12 Non-adhered region
20 Hydrogel laminate
21 Swellable gel thin film layer
22 Non-swellable gel layer
30 Hydrogel channel
40 Sensor section
41 Unit sensor section
42 Unit sensor section row
43 Detection surface
44, 45 Probe
50 Liquid feeding tube

1. A hydrogel channel type device with a sensor, the device comprising:
a solid substrate and a hydrogel laminate located on the solid substrate, wherein
the solid substrate has an adhered region that is adhered to the hydrogel laminate and a non-adhered region that is not adhered to the hydrogel laminate at an interface with the hydrogel laminate,
the hydrogel laminate includes a swellable gel thin film layer on the solid substrate and a non-swellable gel layer laminated on the swellable gel thin film layer,
a hydrogel channel in which the swellable gel thin film layer is separated in the non-adhered region is provided between the solid substrate and the hydrogel laminate, and
a sensor section is provided at the interface between the solid substrate and the hydrogel laminate.

2. The hydrogel channel type device with a sensor according to claim 1, wherein the sensor section includes a detection surface that detects an analyte in the vicinity of the sensor section, and a probe that specifically binds to the analyte.

3. The hydrogel channel type device with a sensor according to claim 1, further comprising a liquid feeding tube connected to the hydrogel channel.
